(19) United States
(12) Patent Application Publication
(10) Pub. No.: US 2010/0048990 A1
(43) Pub. Date: Feb. 25, 2010

(54) ENDOSCOPIC NEEDLE FOR NATURAL ORIFICE TRANSLUMENAL ENDOSCOPIC SURGERY

(75) Inventor: Gregory J. Bakos, Mason, OH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/197,653

(22) Filed: Aug. 25, 2008

(51) Int. Cl. A61B 1/018; A61M 25/10

(52) U.S. Cl. 600/106; 604/103.05

(57) ABSTRACT

A translumenal access device may comprise a catheter, an inflatable member, a hollow needle, a stylet, and a guide wire. The catheter may comprise at least one first lumen and at least one second lumen. The at least one first lumen may be configured to slidably receive the guide wire from the proximal end to the distal end of the catheter. The inflatable member may be mounted near the distal end of the catheter, and may be in fluid communication with second lumen. The hollow needle may be mounted on the distal end of the catheter. The hollow needle may be mounted distal to the inflatable member. The stylet may comprise a third lumen and may be configured to be slidably disposed within the hollow needle. The sylet has at least one extended position and at least one retracted position.

### BACKGROUND

The present application relates to endoscopic needle assemblies and more particularly to an improved endoscopic needle assembly that helps to prevent accidental injury to nearby anatomical structures during tissue penetration. Such tissue penetration may occur when a surgeon uses the endoscopic needle assembly to gain access to the peritoneal cavity using translumenal access procedures.

Access to the abdominal cavity may be required for diagnostic and therapeutic endeavors for a variety of medical and surgical diseases. Historically, abdominal access has required a formal laparotomy to provide adequate exposure. Such procedures, which require incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with bums and skin grafting. Other patients simply do not want to have a scar if it can be avoided.

Minimally invasive procedures are desirable because such procedures can reduce pain and provide relatively quick recovery times as compared with conventional open medical procedures. Many minimally invasive procedures are performed with an endoscope (including without limitation laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. The trocar must pass through several layers of overlapping tissue/muscle before reaching the abdominal cavity.

Still less invasive treatments include those that are performed through insertion of an endoscope through a natural body orifice to a treatment region. Examples of this approach include, but are not limited to, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient (e.g., mouth, anus, vagina) are known as Natural Orifice Translumenal Endoscopic Surgery (NOTES™) procedures. Medical instruments such as endoscopic needles may be introduced through the working channel of a flexible endoscope, which typically has a diameter in the range of about 2.5 to about 4 millimeters.

These minimally invasive surgical procedures have changed some of the major open surgical procedures such as gall bladder removal, or a cholecystectomy, to simple outpatient surgery. Consequently, the patient's recovery time has changed from weeks to days. These types of surgeries are often used for repairing defects or for the removal of diseased tissue or organs from areas of the body such as the abdominal cavity.

An issue typically associated with current endoscopic needles is the risk that nearby organs may be accidentally injured by the endscopic needle. The physician normally cannot see anatomical structures on the distal side of the tissue layers when the endoscopic needle is being pushed through the tissue layers. Therefore, there is a risk that adjacent organs may be accidentally injured by the penetrating needle.

There is a need for an improved endoscopic needle assembly that helps to prevent accidental injury to nearby anatomical structures during tissue penetration.

The foregoing discussion is intended only to illustrate some of the shortcomings present in the field of endoscopic surgery, and the scope of the appended claims should not be limited in this context.

### FIGURES

The novel features of the various embodiments are set forth with particularity in the appended claims. The various embodiments, however, both as to organization and methods of operation, may best be understood by reference to the following description, taken in conjunction with the accompanying drawings as follows.

FIG. 1 is a drawing of a flexible, endoscopic portion of a gastroscope inserted into the upper gastrointestinal tract of a patient.

FIG. 2 is a partial perspective view of the distal portion of an endoscope.

FIG. 3 is a side view of one embodiment of an endoscopic needle assembly.

FIG. 4 is a side view of the endoscopic needle assembly of FIG. 3 with an outer sheath translated proximally.

FIG. 5 is a side view of the endoscopic needle assembly of FIG. 3 with a stylet in a retracted position.

FIG. 6 is a side view of the endoscopic needle assembly of FIG. 3 where an endoscopic needle has penetrated a portion of tissue of the patient.

FIG. 7 is a side view of the endoscopic needle assembly of FIG. 3 where the endoscopic needle has fully penetrated the tissue and a deflated inflatable member has been moved into the tissue opening.

FIG. 8 is a side view of the endoscopic needle assembly of FIG. 3 where the endoscopic needle has fully penetrated the tissue and the inflatable member has been inflated.

FIG. 9A is a side view of the endoscopic needle assembly of FIG. 3 where the inflatable member has been inflated, and a distal portion of the endoscope has been moved distally to the proximal end of the inflatable member.

FIG. 9B is a side view of the endoscopic needle assembly of FIG. 3 where the inflatable member and the distal portion of the endoscope has been moved distally through the tissue.

FIG. 10 is a side view of the endoscopic needle assembly of FIG. 3 with the inflatable member deflated for removal from the patient through the endoscope.

FIG. 11 is a perspective sectional view of one embodiment of a surgical instrument that is adapted to employ the endoscopic needle assembly of FIG. 3 to help prevent injury to nearby anatomical structures during endoscopic needle penetration.

FIG. 12 is a perspective view of one embodiment of a surgical instrument that is adapted to employ the endoscopic needle assembly of FIG. 3.

FIG. 13 is an exploded view of the surgical instrument of FIG. 12.

FIG. 14 is a perspective view of a portion of an endoscopic needleshaft assembly of FIG. 12.

### DESCRIPTION

Before explaining the various embodiments in detail, it should be noted that the embodiments are not limited in their application or use to the details of construction and arrangement of parts illustrated in the accompanying drawings and description. The illustrative embodiments may be implemented or incorporated in other embodiments, variations and modifications, and may be practiced or carried out in various ways. For example, the endoscopic needle assembly configurations disclosed herein are illustrative only and not meant to limit the scope or application thereof. Furthermore, unless otherwise indicated, the terms and expressions employed herein have been chosen for the purpose of describing the illustrative embodiments for the convenience of the reader and are not to limit the scope of the appended claims thereof.

A physician may fully penetrate an endoscopic needle assembly through tissue layers of an organ in order to allow access to the peritoneal cavity of the patient, for example. The physician normally cannot see anatomical structures on the distal side of the tissue layers through the endoscope and therefore may accidentally injure nearby organs with the penetrating needle. An aspect of the endoscopic needle assembly, a veress-type needle configuration, is provided to help prevent such accidental injury.

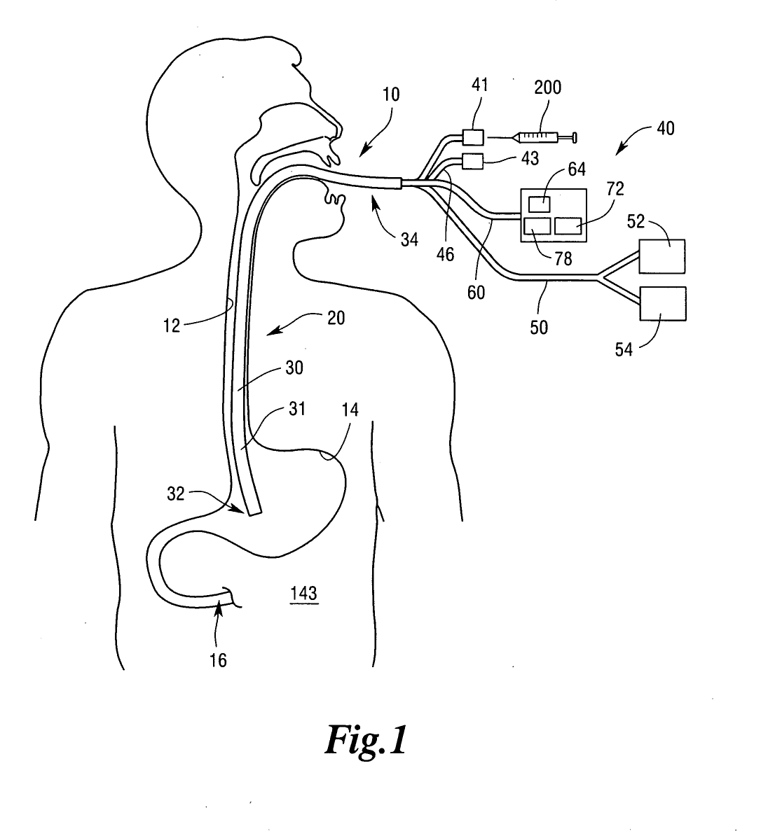

Newer procedures have developed which may even be less invasive than the laparoscopic procedures used in earlier surgical procedures. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as NOTES™. NOTES™ is a translumenal access surgical technique whereby operations can be performed trans-orally (as depicted in FIG. 1), trans-anally, and/or trans-vaginally.

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of the various embodiments is defined solely by the claims. The features illustrated or described in connection with one embodiment may be combined with the features of other embodiments. Such modifications and variations are intended to be included within the scope of the claims.

It will be appreciated that the terms “proximal” and “distal” are used herein with reference to a clinician gripping the surgical instrument. Thus, the endoscopic needle assemblies are distal with respect to the handle assemblies of the surgical instrument. It will be further appreciated that, for convenience and clarity, spatial terms such as “top” and “bottom” also are used herein with respect to the clinician gripping the handle. However, surgical instruments are used in many orientations and positions, and these terms are not intended to be limiting and absolute.

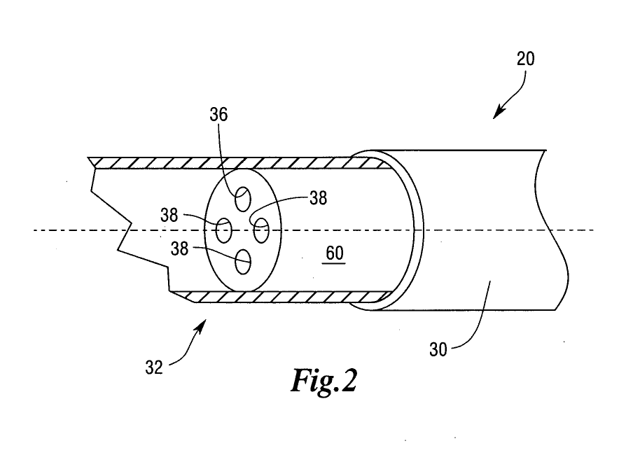

FIG. 1 illustrates a flexible endoscopic portion 31 of a gastroscope inserted into the upper gastrointestinal tract of a patient. FIG. 2 is a drawing of the distal portion 32 of an endoscope. FIG. 1 illustrates, in general form, one embodiment of a surgical instrument 20 that can be inserted through a natural orifice such as the mouth 10 and esophagus 12 into the stomach 14 to establish a surgical opening in the stomach 14 for performing a surgical operation such as a gall bladder removal, or a cholecystectomy. As shown in FIG. 2, the surgical instrument 20 may comprise a hollow outer sleeve 30 that has a distal end 32 and a proximal end 40 (FIG. 1). In various embodiments, the hollow outer sleeve 30 may be fabricated from, for example, nylon or high density polyethylene plastic. In various embodiments, the hollow outer sleeve 30 can serve to define various tool-receiving passages 38 that extend from the natural orifice 10 to the surgical site. In addition, the hollow outer sleeve may serve to define a viewing port 36. An endoscope 60 may be used for viewing a surgical site within the patient's body. Various cameras and/or lighting apparatuses may be inserted into the viewing port 36 of the endoscope to provide the surgeon with a view of the surgical site.

As shown in FIG. 1, in various embodiments, one of the tools or surgical instruments that can be accommodated in the tool-receiving passage 38 is a hollow vacuum/air tube 50 that may communicate with at least one of a vacuum source 52 and a source of pressurized air 54 (FIG. 1). In one embodiment, the vacuum/air tube 50 can be sized to receive therein another surgical instrument in the form of the endoscope 60. A variety of different types of endoscopes are known and, therefore, their specific construction and operation will not be discussed in great detail herein. In various embodiments, the endoscope 60 may operably support a video camera that communicates with a video display unit 64 that can be viewed by the surgeon during the operation. In addition, the endoscope 60 may further have a fluid-supply lumen therethrough that is coupled to a source 72 of water, saline solution, and/or any other suitable fluid and/or an air supply lumen that is coupled to the source of air 78.

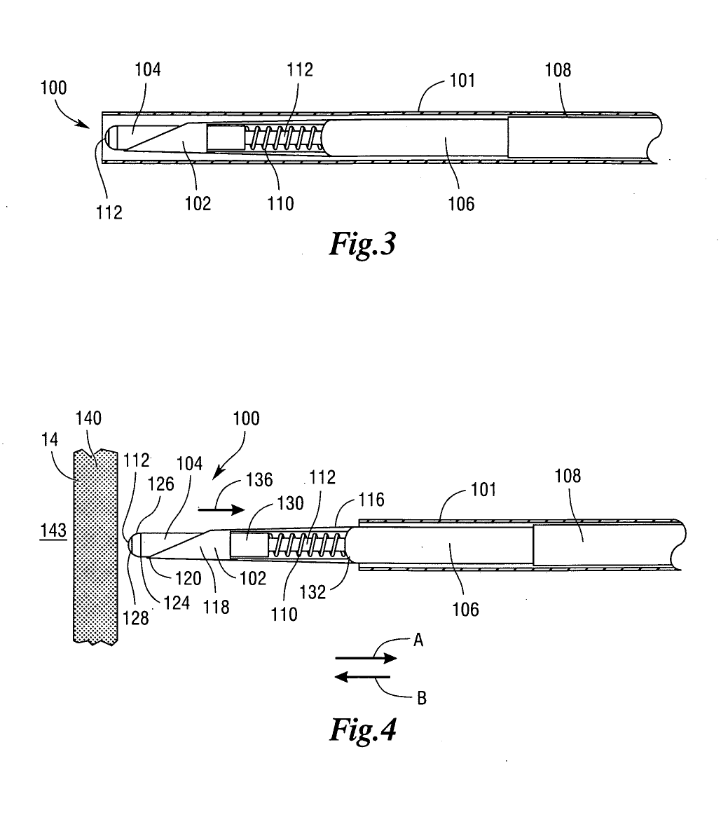

FIG. 3 is a side view of one embodiment of an endoscopic needle assembly. In various embodiments, the endoscope 60 may comprise the one or more working channels 38 (FIG. 2) extending therethrough for receiving various instruments such as the endoscopic needle assembly 100, for example. The endoscopic needle assembly 100 may be configured to be disposed within an outer sheath 101. The endoscopic needle assembly 100 may comprise, for example, an endoscopic needle 102, a needle knife, or other suitable incisor-type instrument that may be inserted through a working channel 38 in the endoscope 60. The outer sheath 101 may be configured to retain the endoscopic needle 102. The endoscopic needle 102 may be attached to the catheter 106 with an adhesive such as cyanoacrylate, epoxy resin, or light activated glue, or any other suitable attachment means. In various other embodiments, the endoscopic needle 102 may be attached to the catheter 106 through welding, bolting, screwing, or any other suitable attachment method.

In various embodiments, the endoscopic needle assembly 100 may comprise a stylet 104, the catheter 106 or cannula, and an inflatable member 108. The outer sheath 101 also may be configured to retain the stylet 104, the catheter 106, and the inflatable member 108. The catheter 106 may be formed from a flexible tube defining a central channel, or lumen, and a secondary channel, or lumen. The central channel of the catheter 106 may be configured to pass from the proximal end of the catheter 106 at or near the endoscope handle to the distal end of the catheter 106. The central channel of the catheter 106 may be further configured to allow a guide wire 112 to extend from the proximal end of the catheter 106 through the distal end of the catheter 106. The secondary channel may extend from the proximal end of the catheter towards the distal end of the catheter 106. The secondary channel may be in fluid communication with the inflatable member 108. The secondary channel may be configured to supply fluid to inflate the inflatable member 108. The inflatable member 108 may comprise an expandable balloon, pouch or bag that extends around, and may be attached to the catheter 106 with an adhesive such as cyanoacrylate, epoxy resin, or light activated glue, or any other suitable attachment means, for example, such that a substantially fluid tight seal is established between the inflatable member 108 and the secondary channel of the catheter 106.

FIG. 4 is a side view of one embodiment of the endoscopic needle assembly 100 of FIG. 3 with the outer sheath 101 translated proximally in the direction indicated by arrow A. In various embodiments, the outer sheath 101 may be translated proximally to expose a portion of the endoscopic needle 102 and a portion of the stylet 104. The inflatable member 108 may be retained in the outer sheath 101 to keep the inflatable member 108 securely retained against the catheter 106. The stylet 104 also may be configured to further translate proximally to expose the inflatable member 108 and at least a portion of the catheter 106. The endoscopic needle 102 may be hollow. The stylet 104 may be configured to be retained within the endoscopic needle 102. The endoscopic needle assembly 100 is shown in FIG. 4 in a shielding, or non-compressed, position with the stylet 104 extending distally past the endoscopic needle 102. This may allow the stylet 104 to contact tissue prior to the endoscopic needle 102 contacting the same tissue. In operation, the outer sheath 101 may be translated proximally to expose at least a portion of the endoscopic needle assembly 100 which may include the stylet 104 and the endoscopic needle 102. Then the endoscopic needle assembly 100 may be pressed against a portion of tissue such that the stylet 104 contacts the tissue. As the force applied to the endoscopic needle assembly 100 by the tissue is increased, the stylet 104 may slidably proximally retract into the endoscopic needle 102, as shown by arrow 136, until the endoscopic needle 102 punctures the tissue 140 and removes the force placed on the endoscopic needle assembly 100. Once the endoscopic needle 102 has punctured the tissue 140, the stylet 104 may return to the shielding position where the stylet 104 extends past the endoscopic needle 102. In the illustrated embodiment, the tissue 140 represents the wall of the stomach 14 (FIG. 1). The endoscopic needle assembly 100 is advanced through the wall of the stomach 14 into the peritoneal cavity 143. It will be appreciated, however, that these procedures may be employed to penetrate any hollow body lumen. Therefore, the illustrative embodiments should not be limited in this context.

In various embodiments, the endoscopic needle 102 may be formed of a tube comprising a channel extending from a proximal end 116 of the endoscopic needle 102 to a distal end 118 of the endoscopic needle 102. The endoscopic needle 102 may taper from a first cross-section at the proximal end 116 to a second, smaller, cross-section at the distal end 118. The distal end 118 of the endoscopic needle 102 may comprise a tissue penetrating tip 120. The endoscopic needle 102 may be ground to form the tissue penetrating tip 120. The endoscopic needle 102 may be fabricated from medical grade stainless steel hypodermic tubing or any other suitable medical grade material, which may include metal and/or plastic suitable for medical, for example, applications. Alternatively, the endoscopic needle 102 may be formed from an alternate type of metallic or polymeric tube and attached to a cannulated needle (not shown), such as by bolting, screwing, welding, crimping, gluing or any other suitable method. The endoscopic needle 102 may have a diameter in the range of 10-35 gage. For example, the endoscopic needle 102 may be formed from 19 gage stainless steel hypodermic tubing having an outer diameter of approximately 0.043 inches (1.09 millimeters) and a wall thickness of approximately 0.003 inches (0.076 millimeters). The stylet 104 may be configured to be slidably disposed within the hollow endoscopic needle 102.

In various embodiments, the stylet 104 may be formed of a tube comprising a channel extending from a proximal end 130 of the stylet 104 to a distal end 124 of the stylet 104. The distal end 124 of the stylet 104 may comprise an exit port 128 and a blunt tip 126. The channel of the stylet 104 may be configured to retain the guide wire 112. The guide wire 112 may extend from the endoscope 60 (FIGS. 1 and 2) through the catheter 106 and the stylet 104 and exit the stylet 104 through the exit port 128. The guide wire 112 may be flexible and may be fabricated from nytenol, or any other suitable material, with a TEFLON®, or any other suitable coating, placed upon the guide wire 112. In one embodiment, the guide wire 112 may be formed from a wire with a diameter in the range of about 0.02 to about 0.04 inches, or any other suitable diameter. The guide wire 112 should be of a diameter large enough to allow the guide wire 112 to move organs and other tissue from the path of the endoscopic needle assembly 100. The stylet 104 may be fabricated from metal, plastic, or any other material suitable for medical applications. The guide wire 112 may be configured to freely move throughout its path from the endoscope 60 to the distal end of the stylet 104. The operator may control the guide wire 112 from the proximal end of the endoscope 60. The operator may extend the guide wire 112 distally in the direction indicated by arrow B to the end of the stylet 104 and out the exit port 128. Alternatively, the operator may retract the guide wire 112 proximally in direction A into the stylet 104. The operator may extend the guide wire 112 out of the exit port 128 to push organs and/or blood vessels out of the path of the needle 102. The guide wire 112 may provide a track for the endoscopic needle 102 to follow so that once the endoscopic needle 102 has punctured the intended tissue 140, the operator may advance the guide wire 112 ahead to help guide the endoscopic needle assembly 100 away from other tissue, organs and/or blood vessels that the operator does not want to puncture. In addition, extending the guide wire 112 beyond the distal end of the stylet 104 provides that the guide wire 112 contacts additional tissue before the stylet 104. Accordingly, the stylet 104 does not retract proximally in direction A and the endoscopic needle 102 remains unexposed to prevent unintended puncture of tissue. The guide wire 112 may be retracted proximally in direction A upon reaching another portion of tissue that requires penetration thus allowing the stylet 104 and the endoscopic needle 102 to interact as previously discussed to puncture the intended tissue.

In various embodiments, a biasing member 110 may be disposed between the proximal end 130 of the stylet 104 and the distal end 132 of the catheter 106. The guide wire 112 may pass through a central opening defined by the biasing member 110. In one embodiment, the biasing member 110 may be secured to the proximal end 130 of the stylet 104 and/or the distal end 132 of the catheter 106 through bolting, welding, gluing, or any other suitable attachment method. In various other embodiments, the biasing member 110 may be secured to the proximal end 130 of the stylet 104 and/or the distal end 132 of the catheter 106 with a pin (not shown) mounted to the proximal end 130 and/or the distal end 132 of the catheter 106. These pins may be configured to be at least partially inserted into the biasing member 110 to keep the biasing member 110 retained in place between the stylet 104 and the catheter 106. One skilled in the art will recognize that these retention methods may be combined. The biasing member 110 may be a coil spring (as shown in FIGS. 3-8), a leaf spring, or any other suitable biasing member.

In various embodiments, the biasing member 110 may apply a predetermined biasing force to bias the stylet 104 to the shielding position. As previously discussed, the stylet 104 can move to the compressed, or retracted, position when the stylet 104 is pushed against the tissue 140 with a force greater than the biasing force, such that the endoscopic needle 102 can penetrate the tissue 140. For example, the biasing member 110 may actuate the needle to extend past the stylet 104 to penetrate the tissue 140 when a specified amount of force is applied to the stylet 104. Once the endoscopic needle 102 has penetrated through the tissue, the stylet 104 can immediately extend to the shielding position to help prevent accidental injury to nearby anatomical structures. In addition, once the endoscopic needle 102 has penetrated the tissue, the guide wire 112 may be extended out of the exit port 128. The biasing member 110 may be fabricated from metal, plastic, or any other material suitable for medical applications.

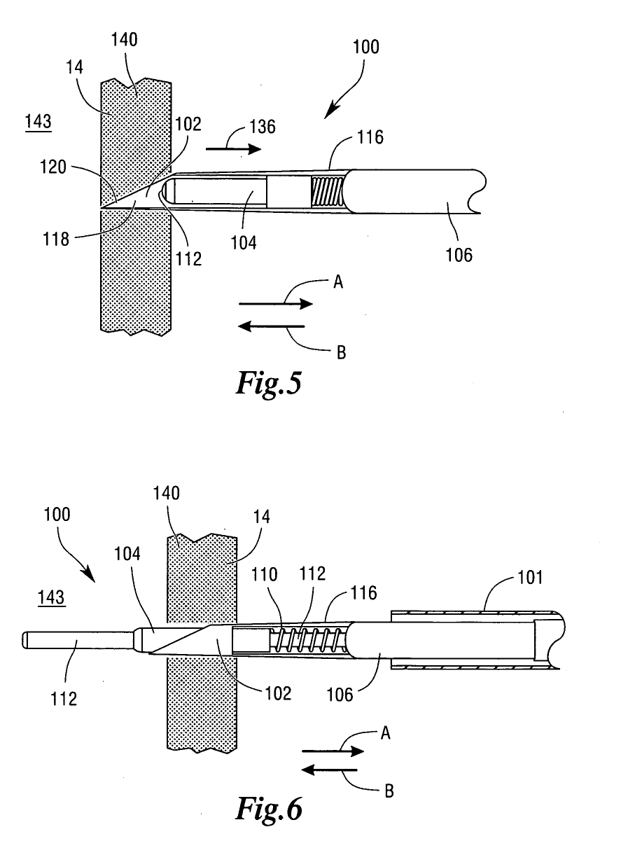

FIG. 5 is a side view of one embodiment of the endoscopic needle assembly 100 of FIG. 3 with the stylet in a retracted position. As previously discussed and illustrated in FIG. 4, the stylet 104 of the endoscopic needle assembly 100 may be placed against the tissue 140 of a patient and then pushed distally in direction B. In various embodiments, the stylet 104 may extend past the distal end of the endoscopic needle 102 in the shielding position. As the endoscopic needle assembly 100 is pushed against the tissue 140, the stylet 104 contacts the tissue 140 before the endoscopic needle 102. As the stylet 104 is pushed against the tissue 140, the stylet 104 may retract into the endoscopic needle 102. As shown in FIG. 5, the stylet 104 may be substantially within the endoscopic needle 102 as the endoscopic needle 102 penetrates the tissue 140.

FIG. 6 is a side view of one embodiment of the endoscopic needle assembly 100 of FIG. 3 with the endoscopic needle 102 penetrating a portion of the tissue 140. In the embodiment illustrated in FIG. 6, once the endoscopic needle 102 has penetrated the tissue 140, the stylet 104 is forced to move distally in direction B past the distal end of the endoscopic needle 102 due to the interaction of the stylet 104 and the biasing member 110. Additionally, as previously discussed, the guide wire 112 also may be extended from the stylet 104 once the endoscopic needle 102 has penetrated the tissue 140. An operator of the surgical instrument may extend the guide wire 112 from the proximal end of the endoscope 60. The operator may choose to extend the guide wire 112 past the distal end 126 of the stylet.

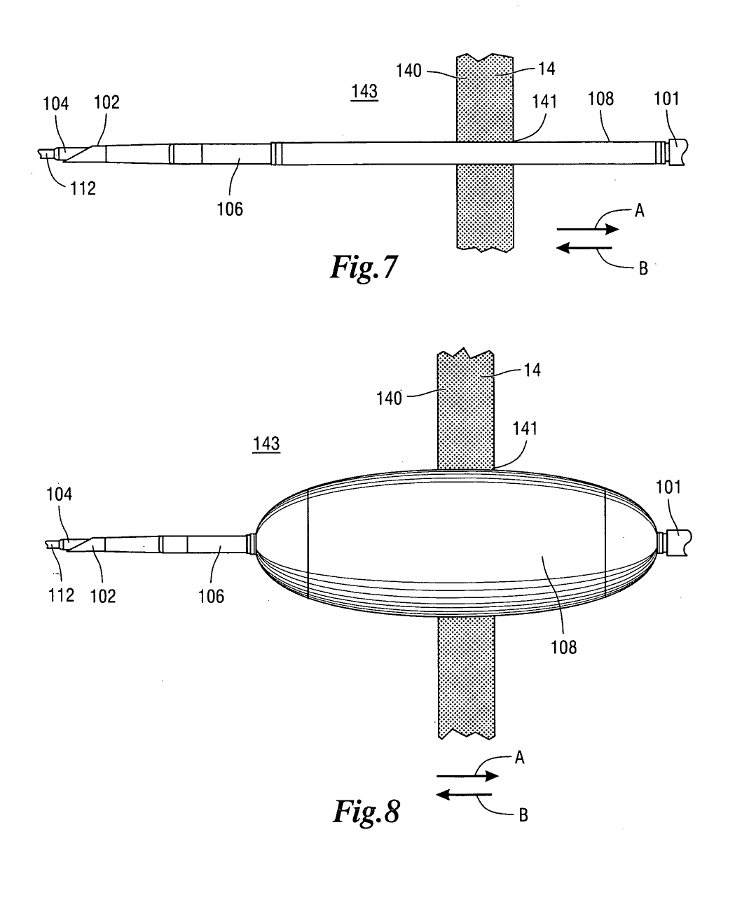

FIG. 7 is a side view of one embodiment of the endoscopic needle assembly 100 of FIG. 3 where the endoscopic needle 102 has fully penetrated the tissue 140 and the deflated inflatable member 108 has been moved distally in direction B into the opening 141 in the tissue 140. As illustrated in FIG. 7, the outer sheath 101 may be further translated proximally to expose the inflatable member 108 and at least a portion of the catheter 106. The outer sheath 101 may be translated proximally in direction A either before or after the inflatable member 108 has been extended distally into the opening 141 formed in the tissue 140 by the endoscopic needle 102. The inflatable member 108 may be extended distally into the opening 141 of the tissue 140 such that approximately half of the inflatable member 108 is located on each side of the tissue 140 wall.

FIG. 8 is a side view of one embodiment of the endoscopic needle assembly 100 of FIG. 3 where the endoscopic needle 102 has fully penetrated the tissue 140 and the inflatable member 108 has been inflated. In one embodiment, the inflatable member 108 may be fabricated from thin films of nylon, polyethylene terephtalate (“PET”), polyurethane plastics, latex elastomers, or any other suitable material. In other embodiments, the inflatable member 108 may be fabricated from a material that is not expandable, but nevertheless is sized to inflate into a desired shape, such as the shape illustrated in FIG. 8, or any other suitable shape. Once the inflatable member 108 has been located into the proper position in the opening 141 in the tissue 140, the inflatable member 108 may be inflated. The inflatable member 108 may be inflated with an inflation syringe 200 (as shown in FIG. 1) or any other suitable arrangement for supplying inflation fluid to the inflatable member 108. The fluid supplied to the inflatable member 108 may comprise air, water, saline solution, or any other suitable inflation fluid.

In one embodiment, the syringe 200 (FIG. 1) may supply fluid to the inflatable member 108 through an inflation port 41 (FIG. 1). The inflation port 41 may be connected to a secondary lumen (not shown) of the catheter 106 in a fluid-tight manner. For example, the inflation port 41 may be connected to the secondary lumen through a distal pressure supply lumen 40 which can be provided through the outer sleeve 30. The distal pressure supply lumen 40 may be attached to the inflation port 41 and the secondary lumen with an adhesive such as cyanoacrylate, epoxy resin, or light activated glue, or any other suitable attachment means, for example, such that a substantially fluid tight seal is established between the inflation port 41 and the secondary lumen.

In various embodiments, a flexible distal check valve flap or sleeve (not shown) can be oriented over the distal pressure supply lumen 40. The flexible distal check valve flap enables the flow of a pressurized fluid medium (e.g., air, water, or saline) of the distal pressure supply lumen 40 and into the inflatable member 108 to inflate the inflatable member 108. In various other embodiments, the check valve flap may comprise a soft rubber or plastic sleeve that is constructed to permit the pressurized medium to enter the inflatable member 108. The port 41 may be coupled to the proximal end of the distal pressure supply lumen 40 to enable the fluid medium to be injected therein by the syringe 200 (FIG. 1), for example. After the inflatable member 108 has been inflated to a desired shape, the flow of pressurized fluid medium into the inflatable member 108 can be discontinued and the pressure in the distal supply lumen 40 may be relieved at the proximal end. When the pressure inside the inflatable member 108 exceeds the pressure in the distal pressure supply lumen 40, the back pressure of the pressurized fluid medium within the inflatable member 108 acts on the check valve sleeve to prevent back flow of the pressurized fluid medium through the port 41.

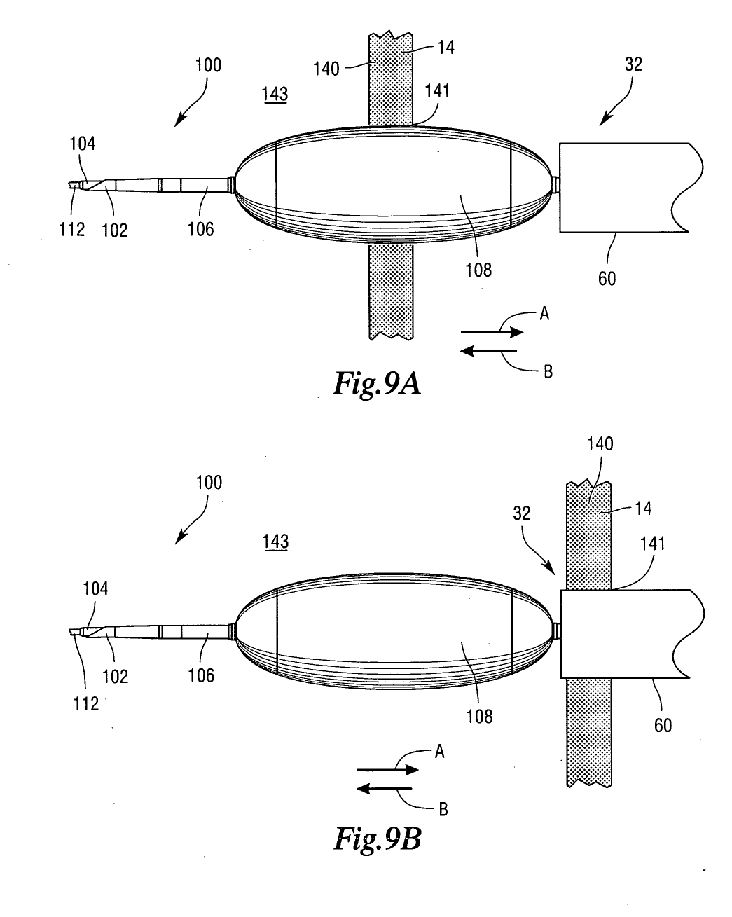

FIG. 9A is a side view of one embodiment of the endoscopic needle assembly 100 of FIG. 3 where the inflatable member 108 has been inflated, and the distal end 32 of the endoscope 60 has been moved distally in direction B to the proximal end of the inflatable member 108. FIG. 9B is a side view of the endoscopic needle assembly 100 where the inflatable member 108 and the distal portion of the endoscope 60 has been moved distally in direction B through the tissue 140. As illustrated in FIG. 9A, once the inflatable member 108 has been inflated the distal end 32 of the endoscope 60 may be moved distally in direction B until the distal end 32 of the endoscope 60 contacts, or nearly contacts, the proximal end of the inflatable member 108. Once the distal end 32 of the endoscope 60 is at or near the proximal end of the inflatable member 108, the endoscope 60 and the endoscopic needle assembly 100 may be pushed distally in direction B through the opening 141 in the tissue 140, as illustrated in FIG. 9B.

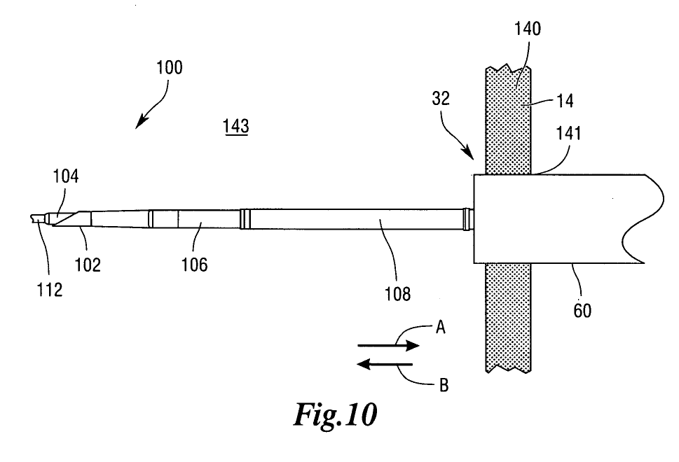

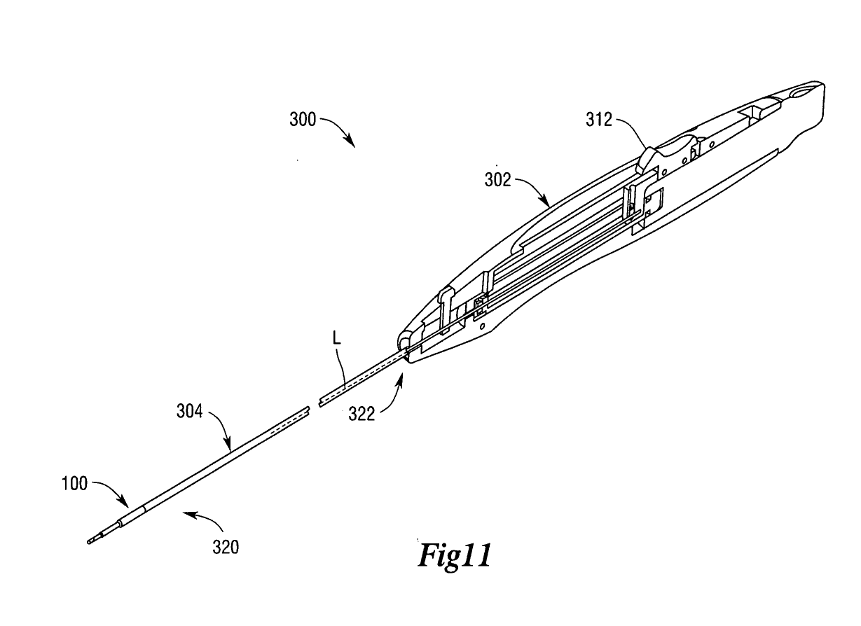

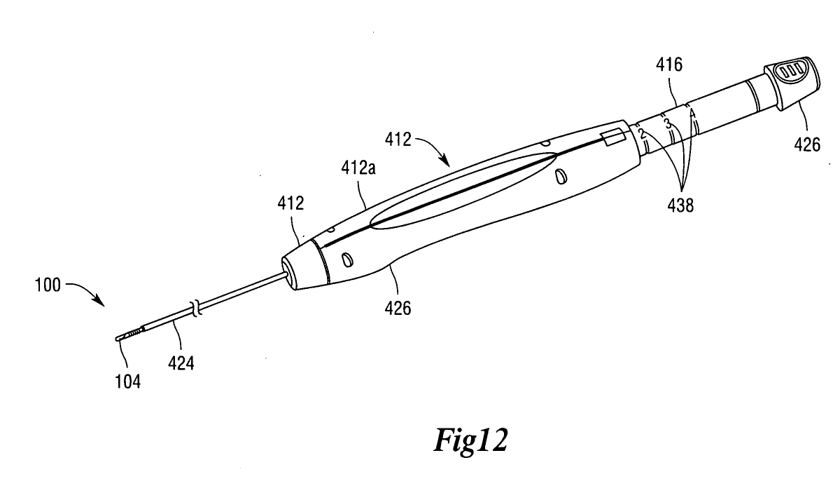

FIG. 10 is a side view of one embodiment of the endoscopic needle assembly 100 of FIG. 3 with the inflatable member 108 deflated for removal from the patient through the endoscope 60. In various embodiments, the endoscopic needle assembly 100 may be removed from the patient through one of the working channels 38 of the endoscope 60 after the endoscope 60 has been pushed through the opening 141 in the tissue 140. In various other embodiments, the guide wire 112 may be used to advance the endoscopic needle assembly 100 through the peritoneal cavity 143 for piercing additional tissue as may be required. The endoscopic needle assembly 100 may be used to puncture a number of tissue instances in the manner previously discussed, and the endoscope 60 may be advanced through those tissue punctures in the manner previously discussed. Once the endoscopic needle assembly 100 has completed its task, the inflatable member 108 may be deflated to fit through one of the working channels 38 of the endoscope 60, and the endoscopic needle assembly 100 may be removed from the working channel 38 to provide other surgical instruments to the surgical site through the working channel 38. The flow of pressurized fluid medium into the inflatable member 108 may be discontinued at this point, and the pressure in the distal supply lumen 40 (FIG. 1) may be relieved through proximal end thereof.

FIG. 11 is a perspective sectional view of one embodiment of a surgical instrument 300 that is adapted to employ the endoscopic needle assembly 100 of FIG. 3 to help prevent injury to nearby anatomical structures during needle penetration. In one embodiment, the surgical instrument 300 comprises an elongate shaft 304 attached to a handle 302. The shaft 304 may be formed of the catheter 106 or may be attached to the catheter 106 through any attachment means such as bolting, screwing, welding, gluing, fusing, or any other suitable method. The shaft 304 comprises a distal end 320 and a proximal end 322 defining a longitudinal axis L therebetween. The shaft 304 may be flexible and may be sized for insertion into any one of the working channels 38 of the flexible endoscope 60 (FIGS. 1 and 2). The surgical instrument 300 may be used in conjunction with any suitable endoscopic needle assembly. The endoscopic needle assembly 100 may be disposed at the distal end 320 of the shaft 304. The endoscopic needle assembly 100 may be attached to the distal end 320 through any attachment means such as bolting, screwing, welding, gluing, fusing, or any other suitable method. The embodiment of the surgical instrument 300 is described next as it may be adapted for use with the endoscopic needle assembly 100, although the surgical instrument 300 also may be adapted for use with various suitable endoscopic needle assemblies and should not be limited in this context. As shown in the embodiments of FIG. 11, the handle 302 comprises an actuator 312. A physician may operate the actuator 312 to deploy guide wire 112 once the endoscopic needle 102 has penetrated the desired tissue.

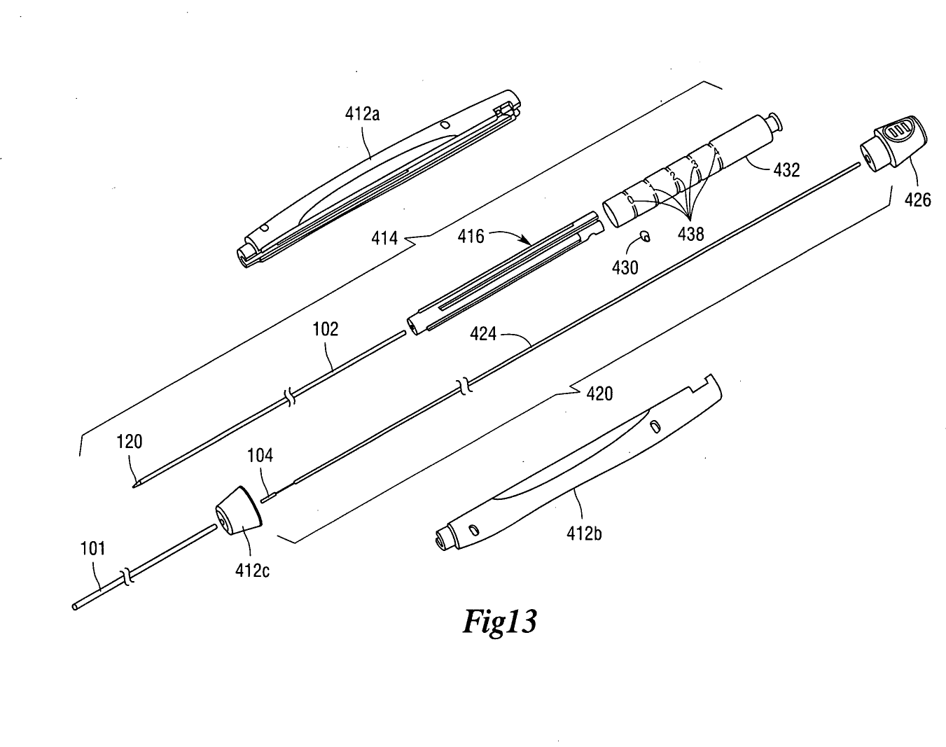

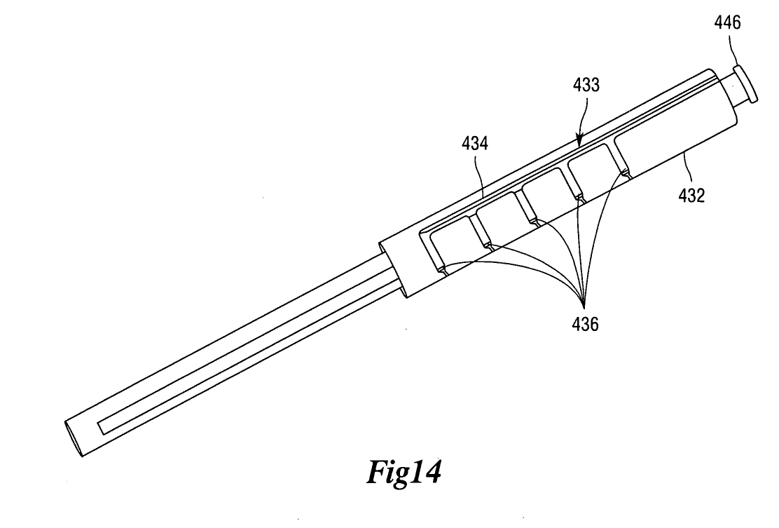

FIG. 12 is a perspective view of one embodiment of a surgical instrument 400 that is adapted to employ the endoscopic needle assembly 100 of FIG. 3. FIG. 13 is an exploded view of the embodiment of the surgical instrument 400 of FIG. 12. FIG. 14 is a perspective view of a portion of an endoscopic needleshaft assembly 414 of the surgical instrument 400 of FIG. 12. In various other embodiments, the surgical instrument 400 generally comprises a handle 412 with the endoscopic needleshaft assembly 414 extending therethrough and extending from a distal end of the handle 412 and is configured to be introduced translumenally. In one embodiment, the endoscopic needleshaft assembly 414 comprises an endoscopic needle shaft 416 slidably disposed within the handle 412. The endoscopic needle 102 comprises the tissue-penetrating tip 120 and extends distally from the endoscopic needle shaft 416. The tissue-penetrating tip 120 may be formed on or coupled to a distal end of the endoscopic needle shaft 416 for penetrating tissue. Although not shown in FIGS. 12-13, a catheter 106 and an inflatable member 108 may be connected to the proximal end of the endoscopic needle 102 and the distal end of the endoscopic needle shaft 416. In one embodiment, the surgical instrument 400 comprises a stylet assembly 420 disposed within the endoscopic needleshaft assembly 414 and is configured to protect the tip 120 until the surgical instrument 400 is positioned against a tissue to be penetrated. The stylet assembly 420 may comprise a stylet shaft 424 extending distally from the handle 412 and is coupled at a proximal end thereof to an end cap 426. The stylet 104 is disposed distal of the distal end of the stylet shaft 424 for protecting the tip 120. The surgical instrument 400 may comprise the outer sheath 101 extending distally from the handle 412. The outer sheath 101 is configured to receive and house the endoscopic needle and stylet shaft assemblies 414, 420 to thereby protect a body lumen, or another instrument in which the surgical instrument 400 may be inserted, from the tissue-penetrating tip 120. As previously discussed, in use, the stylet 104 on the stylet shaft assembly 420 can be positioned relative to the tissue-penetrating tip 120 of the endoscopic needle 102 to render the tip 120 blunt and prevent it from penetrating tissue.

In various embodiments, the handle 412 of the surgical instrument 400 can have any shape and size. The handle 412 may be adapted to facilitate grasping and manipulating the surgical instrument 400. In embodiment illustrated in FIGS. 12-13, the handle 412 has an elongate cylindrical configuration. The handle 412 can be formed from multiple elements, or it can have a unitary configuration. In the illustrated embodiment, the handle 412 comprises two halves 412a, 412b that mate together and house the proximal portions of the endoscopic needle stylet assemblies 414, 420. As shown, a distal end cap 412c can be used to mate the distal ends of the assemblies 414, 420. The end cap 412c, as well as the proximal end of the handle 412, may comprise openings formed therein for receiving the assemblies 414, 420 therethrough.

As previously noted, the surgical instrument 400 also may comprise the outer sheath 101 that houses the distal portion of the endoscopic needle and stylet assemblies 414, 420. The outer sheath 101 can be flexible or rigid. In one embodiment, a distal end of the surgical instrument 400 is adapted to be inserted translumenally, and therefore the outer sheath 101 can be semi-flexible or flexible to allow insertion through a tortuous inner body lumen. As shown in FIGS. 12-13, the outer sheath 101 is fixed to and extends distally from the distal end of the end cap 412c of the handle 412. The length of the outer sheath 101 can vary depending on the intended use of the surgical instrument 400. In the illustrated embodiment, the outer sheath 101 has an elongate length that is adapted for translumenal access. A person skilled in the art will appreciate that in other embodiments the outer sheath 101 of the surgical instrument 400 may be omitted. The handle 412 also may comprise other features, such as a dowel 430 coupled to an inner wall of the handle 412 that is configured to control a position of the tissue-penetrating tip 120 with respect to the handle 412 and the outer sheath 101, as will be discussed in more detail below.

The endoscopic needle shaft assembly 414 of the surgical instrument 400 can have a variety of configurations, and various portions of the endoscopic needle shaft assembly 414 can be flexible or rigid. In one embodiment, a distal end of the endoscopic needle assembly 414, e.g., the endoscopic needle assembly 100, is adapted to be inserted translumenally, and therefore at least portions of the endoscopic needle assembly 100 extending from the handle 412 are semi-flexible or flexible to allow insertion through a tortuous lumen. One skilled in the art will appreciate that the endoscopic needle assembly 100 can be made from a variety of biocompatible materials that have properties sufficient to enable portions of the endoscopic needle assembly 100 extending from the handle 412 to be inserted and moved within channels of a body lumen. The length of the endoscopic needle assembly 100 may vary depending on the intended use of the device, and in one embodiment, the length is adapted for translumenal access. The diameter of the endoscopic needle assembly 100 may vary, and in one embodiment, the diameter is preferably sufficient to slidably receive the stylet 104 of the stylet shaft assembly 420.

In various embodiments, the proximal end of the endoscopic needle shaft assembly 414 may comprise an endoscopic needle shaft 416 coupled to the endoscopic needle assembly 100 of FIG. 3. The endoscopic needle shaft 416 can have a variety of configurations, and in the illustrated embodiment, the endoscopic needle shaft 416 is slidably movable in the handle 412 to allow a position of the tissue-penetrating tip 120 to be adjusted with respect to the outer sheath 101. In particular, movement of the endoscopic needle shaft 416 within the handle 412 can be used to move the tip 120 between a retracted position, in which it is fully disposed within the outer sheath 101, and an extended position, in which the tip 120 extends beyond the distal end of the outer sheath 101. The endoscopic needle shaft assembly 414 can, in other embodiments, be fixedly coupled to or formed integrally with the handle 412.

As shown in FIGS. 12-14, the endoscopic needle shaft 416 may comprise a depth gauge 432 formed on or coupled to a proximal end thereof and adapted to indicate a depth of the tip 120 relative to the outer sheath 101. In one embodiment, the depth gauge 432 may comprise a keyed track 433 formed therein that is adapted to position the tip 120 at various predetermined locations. The keys 436 are radial slots formed along the length of the track 433 and are adapted to receive a dowel 430 which is coupled to an inner wall of the handle 412. The dowel 430 can be locked in the various keys 436 to position the tip 120 relative to the outer sheath 101. In use, the endoscopic needle shaft 416 is rotated to position the dowel 430 within a longitudinal slot 434, and it is moved longitudinally to slide the endoscopic needle shaft assembly 414 relative to the handle 412 to adjust the position of the tissue-penetrating tip 120. After the tip 120 is moved to a desired position, the shaft 416 is rotated to lock the dowel 430 in another key 436 in the track 433 and thereby maintain the endoscopic needle shaft assembly 414 in a fixed position relative to the handle 412 and the outer sheath 101. The depth gauge 432 also may include markings to indicate the depth of the tip 120. As shown, the depth gauge 432 includes five keys 436, and thus five marking 438 (FIGS. 12 and 13) along its length. In the illustrated embodiment, these markings 438 are defined as the values 0-4, but any types of markings to indicate the varying depth levels of the tip 120 are sufficient. A person skilled in the art will appreciate that a variety of other techniques may be used to adjust the depth of the tissue-penetrating tip 120 relative to the outer sheath 101.

In various embodiments, the stylet shaft assembly 420 is disposed within the endoscopic needle shaft assembly 414 and can have a variety of sizes and configurations. In the illustrated embodiment, the stylet shaft assembly 420 comprises the stylet shaft 424 and the stylet 104 that are movably coupled to one another and have a length that allows them to extend through the handle 412 to a position proximal to the distal-most end of the tissue-penetration tip 120 to protect the tip 120 when the surgical instrument 400 is not in contact with tissue. The stylet 104 at the distal end is adapted to protect the tissue-penetrating tip 120 when the device is not in contact with tissue. The shape and size of the stylet 104 may have various configurations, and in the illustrated embodiment, it has a cylindrical configuration with a blunt distal end. The stylet 104 is movable relative to the tissue-penetrating tip 120 between a first position in which the stylet 104 is distal to the tissue-penetrating tip 120 to prevent tissue penetration, and a second position in which the stylet 104 is proximal to, or adjacent to, the tissue-penetrating tip 120 to allow the tip 120 to penetrate tissue. The stylet shaft 424 extends proximally from the stylet 104 and is preferably semi-flexible or flexible to allow insertion through a tortuous lumen.

In various embodiments, the stylet shaft assembly 420 is disposed within the endoscopic needle shaft assembly 414 with the stylet 104 extending adjacent to or distally from the tissue-penetrating tip 120 when the stylet 104 is in the distal position. The needle and stylet shaft assemblies 414, 420 can optionally be releasably attached to each other to allow them to move together with respect to the outer sheath 101 to maintain the position of the stylet 104 with respect to the tip 120. In one embodiment, the stylet shaft 424 may be coupled to an end cap 426, which can releasably mate to the proximal end of the endoscopic needle shaft 416. The endoscopic needle shaft 416 can be coupled to the end cap 426 using a variety of mating techniques, such as a luer lock, threads, a snap fit engagement, an interference fit, and a magnetic engagement.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam.

Although various embodiments have been described herein, many modifications and variations to those embodiments may be implemented. For example, different types of endoscopic needle assemblies may be employed. In addition, combinations of the described embodiments may be used. Also, where materials are disclosed for certain components, other materials may be used. The foregoing description and following claims are intended to cover all such modification and variations. Additional details regarding endoscopic needle assemblies can be found in U.S. patent application Ser. No. 11/380,958 filed on May 1, 2006 entitled “Flexible Endoscopic Safety Needle” to Conlon et al., herein incorporated by reference.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A translumenal access device comprising:
a catheter comprising a proximal end, a distal end, at least one first lumen, and at least one second lumen, the at least one first lumen configured to slidably receive a guide wire from the proximal end to the distal end of the catheter;
an inflatable member mounted near the distal end of the catheter and in fluid communication with the second lumen;
a hollow needle mounted on the distal end of the catheter and mounted distal to the inflatable member;
a stylet comprising a third lumen, the sylet configured to be slidably disposed within the hollow needle, and the sylet comprising at least one extended position and at least one retracted position; and
a guide wire slidably moveable between an extended position and a retracted position, wherein in the extended position, the guide wire is extended distally from the stylet and in the retracted position, the guide wire is retracted proximally from the stylet, and wherein the guide wire is configured to be received in at least a part of the first lumen and at least a part of the third lumen.

2. The translumenal access device of claim 1, wherein the hollow needle comprises a proximal diameter and a distal diameter that is smaller than the proximal diameter, and wherein an outer surface of the needle tapers from the proximal diameter to the distal diameter.

3. The translumenal device of claim 1, wherein the guide wire has an outer diameter between about 0.020 inch to about 0.040 inch.

4. The translumenal access device of claim 1, further comprising a biasing member located within the hollow needle to bias the stylet in an extended position.

5. The translumenal access device of claim 4, wherein the biasing member is disposed between a proximal end of the stylet and the distal end of the catheter.

6. The translumenal access device of claim 5, wherein the biasing member is configured to receive the guide wire through a central opening defined by the biasing member when the guide wire extends from at least a portion of the first lumen into at least a portion of the third lumen.

7. The translumenal access device of claim 1, comprising an outer sheath configured to retain at least one of the hollow needle, the stylet, the biasing member, the guide wire, the inflatable member, and the catheter.

8. The translumenal access device of claim 7, wherein the inflatable member is configured to be in a deflated position when the inflatable member is disposed within the outer sheath.

9. The translumenal access device of claim 7, wherein the inflatable member is configured to be inflated when the inflatable member is removed from the outer sheath.

10. The translumenal access device of claim 9, wherein the second lumen is adapted for fluid communication with a syringe.

11. A surgical instrument having proximal and distal ends defining an axis therebetween, wherein the surgical instrument is flexible and sized for insertion into a working channel of a flexible endoscope, the surgical instrument comprising:
a outer sheath defining a first channel extending from a proximal end of the outer sheath to a distal end of the outer sheath, at least a portion of the outer sheath adapted to retain a catheter, an inflatable member, a needle, a biasing member, a guide wire, and a stylet;
the catheter defining a second channel extending from a proximal end of the catheter to a distal end of the catheter, the second channel adapted to retain the guide wire;
the needle defining a third channel extending from a proximal end of the needle to a distal end of the needle, the needle located at the distal end of the catheter, and the third channel adapted to retain the stylet;
the stylet defining a fourth channel extending from a proximal end of the stylet to a distal end of the stylet, the fourth channel adapted to retain at least a portion of the guide wire;
the biasing member disposed between the style and the catheter; and
the inflatable member retained upon the catheter.

12. The surgical instrument of claim 11, wherein the guide wire is configured to slidably move between an extended position and a retracted position.

13. The surgical instrument of claim 12, wherein in the extended position, the guide wire is extended distally beyond the stylet and in the retracted position, the guidewire is retracted proximally from the stylet.

14. The surgical instrument of claim 11, wherein the biasing member is configured in a decompressed state when the stylet is not pressed against tissue to be penetrated.

15. The surgical instrument of claim 14, wherein the biasing member is adapted to compress when the stylet is pressed against tissue to be penetrated.

16. The surgical instrument of claim 15, wherein the biasing member actuates the needle to extend past the stylet to penetrate tissue when a specified amount of force is applied to the stylet.

17. The surgical instrument of claim 11, wherein the catheter defines an inflation lumen.

18. The surgical instrument of claim 17, wherein the inflation lumen is configured for fluid communication with a syringe to inflate the inflatable member

19. A method comprising:
inserting an endoscope into a lumen of a patient;
inserting a surgical instrument into the lumen of the patient through a working channel of the endoscope;
translating an outer sheath of the surgical instrument proximally to expose at least a portion of a stylet and at least a portion of a needle;
placing a distal portion of the stylet near a portion of tissue to be penetrated;
pressing the surgical instrument against the tissue causing the stylet to retract into the needle;
penetrating the tissue with the needle;
translating the outer sheath further to expose an inflatable member;
inserting the surgical instrument through the penetration in the tissue until the inflatable member extends from one side of the penetration to another side of the penetration;
inflating the inflatable member;
placing a distal end of the endoscope at a proximal end of the inflatable member;
forcing the inflatable member and the distal end of the endoscope through the penetration;
deflating the inflatable member; and
removing the surgical instrument from the working channel of the endoscope.

20. The method of claim 19, further comprising:
sterilizing the surgical instrument; and
storing the surgical instrument in a sterile container.
